(19) United States
(12) Patent Application Publication
(10) Pub. No.: US 2023/0218594 A1
(43) Pub. Date: Jul. 13, 2023

(54) THERAPEUTIC COMPOUNDS, COMPOSITIONS, AND METHODS OF USE THEREOF

(71) Applicant: eXIthera Pharmaceuticals, Inc., Westborough, MA (US)

(72) Inventors: Neil J. Hayward, Westborough, MA (US); Charles V. Pollack, Jr, Westborough, MA (US); Michael A. Kurz, Westborough, MA (US)

(21) Appl. No.: 18/122,519

(22) Filed: Mar. 16, 2023

Related U.S. Application Data
(63) Continuation of application No. PCT/US2021/050623, filed on Sep. 16, 2021.
(60) Provisional application No. 63/079,742, filed on Sep. 17, 2020.
(60) Provisional application No. 63/079,746, filed on Sep. 17, 2020.

(51) Int. Cl. A61K 31/4427; A61K 9/00; A61K 45/06; A61P 9/10

(52) U.S. Cl. CPC A61K 31/4427; A61K 9/0019; A61K 45/06; A61P 9/10

(57) ABSTRACT

Provided herein are methods of treating a subject who has a viral respiratory infection, comprising administering to the subject a therapeutically effective amount of a compound of formula (I):

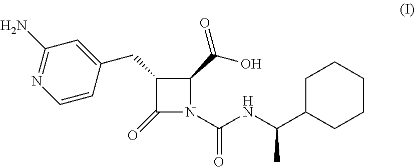 [SMILES: C[C@@H](NC(=O)N1C(=O)[C@H](Cc2ccnc(N)c2)[C@H]1C(=O)O)C1CCCCC1]

or a pharmaceutically acceptable salt thereof.

### CROSS REFERENCE TO RELATED APPLICATIONS

This application is a Continuation of PCT Application No.: PCT/US2021/050623 filed Sep. 16, 2021, which claims priority to and the benefit of U.S. Provisional Patent Application Nos. 63/079,746, filed Sep. 17, 2020 and 63/079,742, filed Sep. 17, 2020, each of which is incorporated herein by reference in its entirety.

### BACKGROUND

Blood coagulation is the first line of defense against blood loss following injury. The blood coagulation “cascade” involves a number of circulating serine protease zymogens, regulatory cofactors and inhibitors. Each enzyme, once generated from its zymogen, specifically cleaves the next zymogen in the cascade to produce an active protease. This process is repeated until finally thrombin cleaves the fibrinopeptides from fibrinogen to produce fibrin that polymerizes to form a blood clot. Although efficient clotting limits the loss of blood at a site of trauma, it also poses the risk of systemic coagulation resulting in massive thrombosis. Under normal circumstances, hemostasis maintains a balance between clot formation (coagulation) and clot dissolution (fibrinolysis). However, in certain disease states such as acute myocardial infarction and unstable angina, the rupture of an established atherosclerotic plaque results in abnormal thrombus formation in the coronary arterial vasculature.

Diseases that stem from blood coagulation, such as myocardial infarction, unstable angina, atrial fibrillation, stroke, pulmonary embolism, and deep vein thrombosis, are among the leading causes of death in developed countries. Current anticoagulant therapies, such as injectable unfractionated (UFH) and low molecular weight (LMW) heparin and orally administered warfarin (coumadin) and direct oral anticoagulants (DOACs) that selectively inhibit thrombin or Factor X/Xa, carry the risk of bleeding episodes and display patient-to-patient variability that results in the need for close monitoring and titration of therapeutic doses. Consequently, there is a large medical need for novel anticoagulation drugs that lack some or all of the side effects of currently available drugs.

Factor XIa is an attractive therapeutic target involved in the pathway associated with these diseases. Increased levels of Factor XIa or Factor XIa activity have been observed in several thromboembolic disorders, including venous thrombosis (Meijers et al., N. Engl. J. Med. 342:696, 2000), acute myocardial infarction (Minnema et al., Arterioscler Thromb Vasc Biol 20:2489, 2000), acute coronary syndrome (Butenas et al., Thromb Haemost 99:142, 2008), coronary artery disease (Butenas et al., Thromb Haemost 99:142, 2008), chronic obstructive pulmonary disease (Jankowski et al., Thromb Res 127:242, 2011), aortic stenosis (Blood Coagul Fibrinolysis, 22:473, 2011), acute cerebrovascular ischemia (Undas et al., Eur J Clin Invest, 42:123, 2012), and systolic heart failure due to ischemic cardiomyopathy (Zabcyk et al., Pol Arch Med Wewn. 120:334, 2010). Patients that lack Factor XI because of a genetic Factor XI deficiency exhibit few, if any, ischemic strokes (Salomon et al., Blood, 111:4113, 2008). At the same time, loss of Factor XIa activity, which leaves one of the pathways that initiate coagulation intact, does not disrupt hemostasis. In humans, Factor XI deficiency can result in a mild-to-moderate bleeding disorder, especially in tissues with high levels of local fibrinolytic activity, such as the urinary tract, nose, oral cavity, and tonsils. Moreover, hemostasis is nearly normal in Factor XI-deficient mice (Gailani, Blood Coagul Fibrinolysis, 8:134, 1997). Furthermore, inhibition of Factor XI has also been found to attenuate arterial hypertension and other diseases and dysfunctions, including vascular inflammation (Kossmann et al. Sci. Transl. Med. 2017, Vol. 9, Issue 375, Abstract aah4923).

Consequently, compounds that inhibit Factor XIa have the potential to prevent or treat a wide range of disorders while avoiding the side effects and therapeutic challenges that plague drugs that inhibit other components of the coagulation pathway. Moreover, due to the limited efficacy and adverse side effects of some current therapeutics for the inhibition of undesirable thrombosis (e.g., deep vein thrombosis, hepatic vein thrombosis, and stroke), improved compounds and methods (e.g., those associated with Factor XIa) are needed for preventing or treating undesirable thrombosis.

Another therapeutic target is the enzyme kallikrein. Human plasma kallikrein is a serine protease that may be responsible for activating several downstream factors (e.g., bradykinin and plasmin) that are critical for coagulation and control of e.g., blood pressure, inflammation, and pain. Kallikreins are expressed e.g., in the prostate, epidermis, and the central nervous system (CNS) and may participate in e.g., the regulation of semen liquefaction, cleavage of cellular adhesion proteins, and neuronal plasticity in the CNS. Moreover, kallikreins may be involved in tumorigenesis and the development of cancer and angioedema, e.g., hereditary angioedema. Overactivation of the kallikrein-kinin pathway can result in a number of disorders, including angioedema, e.g., hereditary angioedema (Schneider et al., J. Allergy Clin. Immunol. 120:2, 416, 2007). To date, there are limited treatment options for HAE (e.g., WO2003/076458).

### SUMMARY

The present invention relates, in part, to methods of treating a subject who has a viral respiratory infection, comprising administering to the subject a compound, or a pharmaceutically acceptable salt thereof, or a pharmaceutical composition disclosed herein. The subject may be at risk of a thromboembolic or thromboinflammatory complication.

Thus, in an aspect, provided herein is a method of treating a subject who has a viral respiratory infection, the method comprising administering to the subject a therapeutically effective amount of a compound of formula (I):

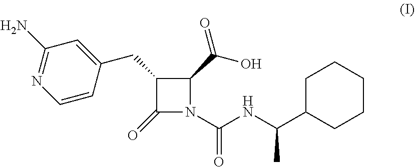 [SMILES: C[C@@H](NC(=O)N1C(=O)[C@H](Cc2ccnc(N)c2)[C@H]1C(=O)O)C1CCCCC1]

or a pharmaceutically acceptable salt thereof.

In another aspect, provided herein is a method of preventing thromboembolic or thromboinflammatory complications in a subject who has a viral respiratory infection, the method comprising administering to the subject a therapeutically effective amount of a compound of formula (I):

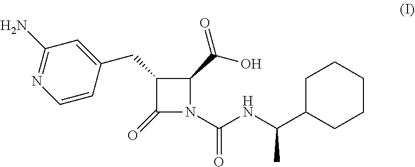 [SMILES: C[C@@H](NC(=O)N1C(=O)[C@H](Cc2ccnc(N)c2)[C@H]1C(=O)O)C1CCCCC1]

or a pharmaceutically acceptable salt thereof.

In another aspect, provided herein is a method of method of reducing the risk of thromboembolic or thromboinflammatory complications in a subject who has a viral respiratory infection, the method comprising administering to the subject a therapeutically effective amount of a compound of formula (I):

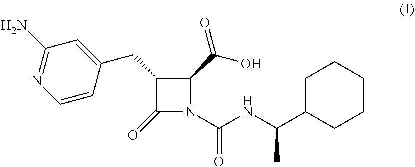 [SMILES: C[C@@H](NC(=O)N1C(=O)[C@H](Cc2ccnc(N)c2)[C@H]1C(=O)O)C1CCCCC1]

or a pharmaceutically acceptable salt thereof.

### DETAILED DESCRIPTION

### Definitions

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition. In an alternate embodiment, the present invention contemplates administration of the compounds of the present invention as a prophylactic before a subject begins to suffer from the specified disease, disorder or condition.

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

Disease, disorder, and condition are used interchangeably herein.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. In some embodiments, the pediatric subject is between the age of 0 and 18 years old. In some embodiments, the adult subject is beyond 18 years old. A contemplated subject is at risk of a thromboembolic or thromboinflammatory complication. In some embodiments, the subject has a viral respiratory infection.

As used herein, the term “artificial surface” refers to any non-human or non-animal surface that comes into contact with blood of the subject, for example, during a medical procedure. It can be a vessel for collecting or circulating blood of a subject outside the subject's body. It can also be a stent, valve, intraluminal catheter or a system for pumping blood. By way of non-limiting example such artificial surfaces can be steel, any type of plastic, glass, silicone, rubber, etc. In some embodiments, the artificial surface is exposed to at least 50%. 60%, 70% 80%, 90% or 100% of the blood of subject.

As used herein, the term “conditioning” or “conditioned” with respect to an artificial surface refers to priming or flushing the artificial surface (e.g., extracorporeal surface) with a compound described herein (e.g., Compound 1) or a pharmaceutically acceptable salt thereof, already in a priming or flushing solution (e.g., blood, a saline solution, Ringer's solution) or as a separate administration to the artificial surface prior to, during, or after a medical procedure.

### Compounds

Described herein are compounds that inhibit Factor XIa or kallikrein. Thus, in one aspect, provided herein is a compound of Formula (I):

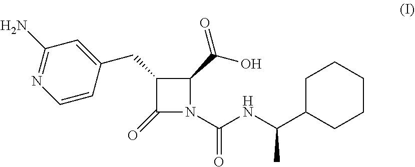 [SMILES: C[C@@H](NC(=O)N1C(=O)[C@H](Cc2ccnc(N)c2)[C@H]1C(=O)O)C1CCCCC1]

or a pharmaceutically acceptable salt thereof.

In some embodiments, a compound of Formula (I) is referred to herein as Compound 1.

In some embodiments, a compound described herein (e.g., Compound 1) is formed into a salt. In some embodiments, the pharmaceutically acceptable salt of Compound 1 is HCl salt of Compound 1.

A compound described herein can be administered as a free acid, a zwitterion or as a salt. A salt can also be formed between a cation and a negatively charged substituent on a compound described herein. Suitable cationic counterions include sodium ions, potassium ions, magnesium ions, calcium ion, and ammonium ions (e.g., a tetraalkyl ammonium cation such as tetramethylammonium ion). In compounds including a positively charged substituent or a basic substituent, a salt can be formed between an anion and a positively charged substituent (e.g., amino group) or basic substituent (e.g., pyridyl) on a compound described herein. Suitable anions include chloride, bromide, iodide, sulfate, nitrate, phosphate, citrate, methanesulfonate, trifluoroacetate, and acetate.

Pharmaceutically acceptable salts of the compounds described herein (e.g., a pharmaceutically acceptable salt of Compound 1) also include those derived from pharmaceutically acceptable inorganic and organic acids and bases. Examples of suitable acid salts include acetate, 4-acetamidobenzoate, adipate, alginate, 4-aminosalicylate, aspartate, ascorbate, benzoate, benzenesulfonate, bisulfate, butyrate, citrate, camphorate, camphorsulfonate, carbonate, cinnamate, cyclamate, decanoate, decanedioate, 2,2-dichloroacetate, digluconate, dodecylsulfate, ethanesulfonate, ethane-1,2-disulfonate, formate, fumarate, galactarate, glucoheptanoate, gluconate, glucoheptonate, glucoronate, glutamate, glutarate, glycerophosphate, glycolate, hemisulfate, heptanoate, hexanoate, hippurate, hydrochloride, hydrobromide, hydroiodide, 1-hydroxy-2-naphthoate, 2-hydroxyethanesulfonate, isobutyrate, lactate, lactobionate, laurate, malate, maleate, malonate, mandelate, methanesulfonate, naphthalene-1,5-disulfonate, 2-naphthalenesulfonate, nicotinate, nitrate, octanoate, oleate, oxalate, 2-oxoglutarate, palmitate, palmoate, pectinate, 3-phenylpropionate, phosphate, phosphonate, picrate, pivalate, propionate, pyroglutamate, salicylate, sebacate, succinate, stearate, sulfate, tartrate, thiocyanate, toluenesulfonate, tosylate, and undecanoate.

Salts derived from appropriate bases include alkali metal (e.g., sodium), alkaline earth metal (e.g., magnesium), ammonium and (alkyl)₄N⁺ salts. This invention also envisions the quaternization of any basic nitrogen-containing groups of the compounds disclosed herein. Water or oil-soluble or dispersible products may be obtained by such quaternization.

As used herein, the compounds of this invention, including the Compound 1, are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention which, upon administration to a recipient, is capable of providing (directly or indirectly) a compound of this invention. Particularly favored derivatives and prodrugs are those that increase the bioavailability of the compounds of this invention when such compounds are administered to a mammal (e.g., by allowing an orally administered compound to be more readily absorbed into the blood), or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of formulae described herein.

Any formula or a compound described herein is also intended to represent unlabeled forms as well as isotopically labeled forms of the compounds, isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, 13C, ¹⁴C, ¹⁵N, ¹⁸F ⁵¹P, ³²P, ³⁵S, ³⁶Cl, ¹²⁵I respectively. The invention includes various isotopically labeled compounds as defined herein, for example, those into which radioactive isotopes, such as ³H, ¹³C, and ¹⁴C are present. Such isotopically labelled compounds are useful in metabolic studies (with ¹⁴C), reaction kinetic studies (with, for example ′H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies, isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of a formula described herein. The concentration of such a heavier isotope, specifically deuterium, may be defined by the isotopic enrichment factor. The term “isotopic enrichment factor” as used herein means the ratio between the isotopic abundance and the natural abundance of a specified isotope If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 8633.3 (99.5% deuterium incorporation).

Isotopically-labelled compounds described herein can generally be prepared by conventional techniques known to those skilled in the art or by processes using an appropriate isotopically-labeled reagents in place of the non-labeled reagent previously employed. Pharmaceutically acceptable solvates in accordance with the invention include those wherein the solvent of crystallization may be isotopically substituted, e.g, D₂O, D₆-acetone, D₆-DMSO. Any asymmetric atom (e.g., carbon or the like) of the compound(s) of the present invention can be present in racemic or enantiomerically enriched, for example the (R)— (S)— or (RS)— configuration, in certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)— or (S)— configuration. Substituents at atoms with unsaturated bonds may, if possible, be present in cis-(Z)- or trans-(E)- form. Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof Any resulting mixtures of isomers can be separated on the basis of the physicochemical differences of the constituents, into the pure or substantially pure geometric or optical isomers, diastereomers, racemates, for example, by chromatography or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. An acidic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, (+)-O,O′-Di-p-toluoyl-D-tartaric acid, mandelic acid, malic acid or camphor-10-sulfonic acid. Racemic products can also be resolved by chiral chromatography, e.g., high pressure liquid chromatography (HPLC) using a chiral adsorbent.

The compounds described herein (e.g., Compound 1) may also be represented in multiple tautomeric forms. In such instances, the invention expressly includes all tautomeric forms of the compounds described herein.

### Methods of Treatment, Prophylaxis, or Reduction of Risk

The compounds described herein (e.g., Compound 1) can inhibit Factor XIa or kallikrein. As a result, these compounds can be useful in the treatment, prophylaxis, or reduction in the risk of a disorder described herein. For example, the compounds described herein (e.g., Compound 1) may be useful in preventing or reducing the risk of thromboembolic or thromboinflammatory complications.

Exemplary disorders include thrombotic events associated with coronary artery and cerebrovascular disease, venous or arterial thrombosis, coagulation syndromes, ischemia (e.g., coronary ischemia) and angina (stable and unstable), deep vein thrombosis (DVT), hepatic vein thrombosis, disseminated intravascular coagulopathy, Kasabach-Merritt syndrome, pulmonary embolism, myocardial infarction (e.g., ST-elevation myocardial infarction or non-ST-elevation myocardial infarction (e.g., non-ST-elevation myocardial infarction before catheterization), cerebral infarction, cerebral thrombosis, transient ischemic attacks, atrial fibrillation (e.g., non-valvular atrial fibrillation), cerebral embolism, thromboembolic complications of surgery (e.g., hip or knee replacement, orthopedic surgery, cardiac surgery, lung surgery, abdominal surgery, or endarterectomy) and peripheral arterial occlusion and may also be useful in treating or preventing myocardial infarction, stroke (e.g., large vessel acute ischemic stroke), angina and other consequences of atherosclerotic plaque rupture. The compounds of the invention possessing Factor XIa or kallikrein inhibition activity may also be useful in preventing thromboembolic disorders, e.g., venous thromboembolisms, in cancer patients, including those receiving chemotherapy and/or those with elevated lactase dehydrogenase (LDH) levels, and to prevent thromboembolic events at or following tissue plasminogen activator-based or mechanical restoration of blood vessel patency. The compounds of the invention possessing Factor XIa or kallikrein inhibition activity may also be useful as inhibitors of blood coagulation such as during the preparation, storage and fractionation of whole blood. Additionally, the compounds described herein may be used in acute hospital settings or periprocedurally, where a patient is at risk of a thromboembolic disorder or complication, and also in patients who are in a heightened coagulation state, e.g., cancer patients.

Factor XIa inhibition, according to the present invention, can be a more effective and safer method of inhibiting thrombosis compared to inhibiting other coagulation serine proteases such as thrombin or Factor Xa. Administration of a small molecule Factor XIa inhibitor should have the effect of inhibiting thrombin generation and clot formation with no or substantially no effect on bleeding times and little or no impairment of haemostasis. These results differ substantially from that of other “direct acting” coagulation protease inhibitors (e.g., active-site inhibitors of thrombin and Factor Xa), which demonstrate prolongation of bleeding time and less separation between antithrombotic efficacy and bleeding time prolongation. A preferred method according to the invention comprises administering to a mammal a pharmaceutical composition containing at least one compound of the invention.

The compounds described herein (e.g., Compound 1) can inhibit kallikrein. As a result, these compounds can be useful in the treatment, prophylaxis, or reduction in the risk of diseases involved in inflammation, such as edema (e.g., cerebral edema, macular edema, and angioedema (e.g., hereditary angioedema)). In some embodiments, the compounds of the invention can be useful in the treatment or prevention of hereditary angioedema. The compounds described herein (e.g., Compound 1) can also be useful in the treatment, prophylaxis, or reduction in the risk of, e.g., stroke, ischemia (e.g., coronary ischemia), and perioperative blood loss for example, Compound 1. The methods of the present invention are useful for treating or preventing those conditions which involve the action of Factor XIa or kallikrein. Accordingly, the methods of the present invention are useful in treating consequences of atherosclerotic plaque rupture including cardiovascular diseases associated with the activation of the coagulation cascade in thrombotic or thrombophilic states.

More particularly, the methods of the present invention can be used in the treatment, prophylaxis, or reduction in the risk of acute coronary syndromes such as coronary artery disease, myocardial infarction, unstable angina (including crescendo angina), ischemia (e.g., ischemia resulting from vascular occlusion), and cerebral infarction. The methods of the present invention further may be useful in the treatment, prophylaxis, or reduction in the risk of stroke (e.g., large vessel acute ischemic stroke) and related cerebral vascular diseases (including cerebrovascular accident, vascular dementia, and transient ischemic attack); venous thrombosis and thrombo-embolism, such as deep vein thrombosis (DVT) and pulmonary embolism; thrombosis associated with atrial fibrillation, ventricular enlargement, dilated cardiac myopathy, or heart failure; peripheral arterial disease and intermittent claudication; the formation of atherosclerotic plaques and transplant atherosclerosis; restenosis following arterial injury induced endogenously (by rupture of an atherosclerotic plaque), or exogenously (by invasive cardiological procedures such as vessel wall injury resulting from angioplasty or post-cranial artery stenting); disseminated intravascular coagulopathy, Kasabach-Merritt syndrome, cerebral thrombosis, and cerebral embolism.

Additionally, the methods of the present invention can be used in the treatment, prophylaxis (e.g., preventing), or reduction in the risk of thromboembolic consequences or complications associated with cancer, thrombectomy, surgery (e.g., hip replacement, orthopedic surgery), endarterectomy, introduction of artificial heart valves, peripheral vascular interventions (e.g., of the limbs), cerebrovascular interventions, large bore interventions used in the treatment of aneurysms, vascular grafts, mechanical organs, and implantation (e.g., trans-catheter aortic valve implantation) or transplantation of organs, (e.g., transplantation of the liver), tissue, or cells); percutaneous coronary interventions; catheter ablation; hemophilia therapy; hemodialysis; medications (such as tissue plasminogen activator or similar agents and surgical restoration of blood vessel patency) in patients suffering myocardial infarction, stroke (e.g., large vessel acute ischemic stroke), pulmonary embolism and like conditions; medications (such as oral contraceptives, hormone replacement, and heparin, e.g., for treating heparin-induced thrombocytopenia); sepsis (such as sepsis related to disseminated intravascular coagulation); pregnancy or childbirth; and another chronic medical condition. The methods of the present invention may be used to treat thrombosis due to confinement (e.g., immobilization, hospitalization, bed rest, or limb immobilization, e.g., with immobilizing casts, etc.). In some embodiments, the thromboembolic consequence or complication is associated with a percutaneous coronary intervention.

Additionally, the compounds described herein (e.g., Compound 1) or pharmaceutically acceptable salts thereof or compositions thereof can be useful in the treatment, prophylaxis and reduction in the risk of a thromboembolic disorder, e.g., a venous thromboembolism, deep vein thrombosis or pulmonary embolism, or associated complication in a subject, wherein the subject is exposed to an artificial surface. The artificial surface can contact the subject's blood, for example, as an extracorporeal surface or that of an implantable device. Such artificial surfaces include, but are not limited to, those of dialysis catheters, cardiopulmonary bypass circuits, artificial heart valves, e.g., mechanical heart valves (MHVs), ventricular assist devices, small caliber grafts, central venous catheters, extracorporeal membrane oxygenation (ECMO) apparatuses. Further, the thromboembolic disorder or associated complication may be caused by the artificial surface or associated with the artificial surface. For example, foreign surfaces and various components of mechanical heart valves (MHVs) are pro-thrombotic and promote thrombin generation via the intrinsic pathway of coagulation. Further, thrombin and FXa inhibitors are contraindicated with thromboembolic disorders or associated complications caused by artificial surfaces such as those MHVs, as these inhibitors are ineffective at blocking the intrinsic pathway at plasma levels that will not cause heavy bleeding. The compounds of the present invention, which can be used as, for example, Factor XIa inhibitors, are thus contemplated as alternative therapeutics for these purposes.

The compounds described herein (e.g., Compound 1) or pharmaceutically acceptable salts thereof or compositions thereof can also be useful for the treatment, prophylaxis, or reduction in the risk of atrial fibrillation in a subject in need thereof. For example, the subject can have a high risk of developing atrial fibrillation. The subject can also in need of dialysis, such as renal dialysis. The compounds described herein (e.g., Compound 1) or pharmaceutically acceptable salts thereof or compositions thereof can be administered before, during, or after dialysis. Direct oral anticoagulants (DOACs) currently available on the market, such as certain FXa or thrombin inhibitors, are contraindicated for atrial fibrillation under such a condition. The compounds of the present invention, which can be used as, for example, Factor XIa inhibitors, are thus contemplated as alternative therapeutics for these purposes. Additionally, the subject can be at a high risk of bleeding. In some embodiments, the subject can have end-stage renal disease. In other cases, the subject is not in need of dialysis, such as renal dialysis. Further, the atrial fibrillation can be associated with another thromboembolic disorder such as a blood clot.

Furthermore, the compounds described herein (e.g., Compound 1) or pharmaceutically acceptable salts thereof or compositions thereof can be used in the treatment, prophylaxis, or reduction in the risk of hypertension, e.g., arterial hypertension, in a subject. In some embodiments, the hypertension, e.g., arterial hypertension, can result in atherosclerosis. In some embodiments, the hypertension can be pulmonary arterial hypertension.

Furthermore, the compounds described herein (e.g., Compound 1) or pharmaceutically acceptable salts thereof or compositions thereof can be used in the treatment, prophylaxis, or reduction in the risk of disorders such as heparin-induced thrombocytopenia, heparin-induced thrombocytopenia thrombosis, or thrombotic microangiopathy, e.g., hemolytic uremic syndrome (HUS) or thrombotic thrombocytopenic purpura (TTP).

In some embodiments, the subject is sensitive to or has developed sensitivity to heparin. Heparin-induced thrombocytopenia (HIT) is the development of (a low platelet count), due to the administration of various forms of heparin. HIT is caused by the formation of abnormal antibodies that activate platelets. HIT can be confirmed with specific blood tests. In some embodiments, the subject is resistant to or has developed resistance to heparin. For example, activated clotting time (ACT) test can be performed on the subject to test for sensitivity or resistance towards heparin. The ACT test is a measure of the intrinsic pathway of coagulation that detects the presence of fibrin formation. A subject who is sensitive and/or resistant to standard dose of heparin typically do not reach target anticoagulation time. Common correlates of heparin resistance include, but are not limited to, previous heparin and/or nitroglycerin drips and decreased antithrombin III levels. In some embodiments, the subject has previously been administered an anticoagulant (e.g. bivalirudin/Angiomax).

The compounds described herein (e.g., Compound 1) or pharmaceutically acceptable salts thereof or compositions thereof can be used to reduce inflammation in a subject. In some embodiments, the inflammation can be vascular inflammation. In some embodiments, the vascular inflammation can be accompanied by atherosclerosis. In some embodiments, the vascular inflammation can be accompanied by a thromboembolic disease in the subject. In some embodiments, the vascular inflammation can be angiotensin II-induced vascular inflammation.

The compounds described herein (e.g., Compound 1) or pharmaceutically acceptable salts thereof or compositions thereof can be used in the treatment, prophylaxis, or reduction in the risk of renal disorders or dysfunctions, including end-stage renal disease, hypertension-associated renal dysfunction in a subject, kidney fibrosis, and kidney injury.

The methods of the present invention may also be used to maintain blood vessel patency, for example, in patients undergoing thrombectomy, transluminal coronary angioplasty, or in connection with vascular surgery such as bypass grafting, arterial reconstruction, atherectomy, vascular grafts, stent patency, and organ, tissue or cell implantation and transplantation. The inventive methods may be used to inhibit blood coagulation in connection with the preparation, storage, fractionation, or use of whole blood. For example, the inventive methods may be used in maintaining whole and fractionated blood in the fluid phase such as required for analytical and biological testing, e.g., for ex vivo platelet and other cell function studies, bioanalytical procedures, and quantitation of blood-containing components, or for maintaining extracorporeal blood circuits, as in a renal replacement solution (e.g., hemodialysis) or surgery (e.g., open-heart surgery, e.g., coronary artery bypass surgery). In some embodiments, the renal replacement solution can be used to treat patients with acute kidney injury. In some embodiments, the renal replacement solution can be continuous renal replacement therapy.

In addition, the methods of the present invention may be useful in treating and preventing the prothrombotic complications of cancer. The methods may be useful in treating tumor growth, as an adjunct to chemotherapy, for preventing angiogenesis, and for treating cancer, more particularly, cancer of the lung, prostate, colon, breast, ovaries, and bone.

### Extracorporeal Membrane Oxygenation (ECMO)

“Extracorporeal membrane oxygenation” (or “ECMO”) as used herein, refers to extracorporeal life support with a blood pump, artificial lung, and vascular access cannula, capable of providing circulatory support or generating blood flow rates adapted to support blood oxygenation, and optionally carbon dioxide removal. In venovenous ECMO, extracorporeal gas exchange is provided to blood that has been withdrawn from the venous system; the blood is then reinfused to the venous system. In venoarterial ECMO, gas exchange is provided to blood that is withdrawn from the venous system and then infused directly into the arterial system to provide partial or complete circulatory or cardiac support. Venoarterial ECMO allows for various degrees of respiratory support.

As used herein, “extracorporeal membrane oxygenation” or “ECMO” refers to extracorporeal life support that provides circulatory support or generates blood flow rates adequate to support blood oxygenation. In some embodiments, ECMO comprises removal of carbon dioxide from a subject's blood. In some embodiments, ECMO is performed using an extracorporeal apparatus selected from the group consisting of a blood pump, artificial lung, and vascular access cannula.

As used herein, “venovenous ECMO” refers to a type of ECMO in which blood is withdrawn from the venous system of a subject into an ECMO apparatus and subjected to gas exchange (including oxygenation of the blood), followed by reinfusion of the withdrawn blood into the subject's venous system. As used herein, “venoarterial ECMO” refers to a type of ECMO in which blood is withdrawn from the venous system of a subject into an ECMO apparatus and subjected to gas exchange (including oxygenation of the blood), followed by infusion of the withdrawn blood directly into the subject's arterial system. In some embodiments, venoarterial ECMO is performed to provide partial circulatory or cardiac support to a subject in need thereof. In some embodiments, venoarterial ECMO is performed to provide complete circulatory or cardiac support to a subject in need thereof.

The compounds of the present invention can be used in the treatment, prophylaxis, or reduction in the risk of a thromboembolic disorder in a subject in need thereof, wherein the subject is exposed to an artificial surface such as that of an extracorporeal membrane oxygenation (ECMO) apparatus (vide supra), which can be used as a rescue therapy in response to cardiac or pulmonary failure. The surface of an ECMO apparatus that directly contacts the subject can be a pro-thrombotic surface that can result in a thromboembolic disorder such as a venous thromboembolism, e.g., deep vein thrombosis or pulmonary embolism, leading to difficulties in treating a patient in need of ECMO. Clots in the circuit are the most common mechanical complication (19%). Major clots can cause oxygenator failure, and pulmonary or systemic emboli.

ECMO is often administered with a continuous infusion of heparin as an anticoagulant to counter clot formation. However, cannula placement can cause damage to the internal jugular vein, which causes massive internal bleeding. Bleeding occurs in 30-40% of patients receiving ECMO and can be life-threatening. This severe bleeding is due to both the necessary continuous heparin infusion and platelet dysfunction. Approximately 50% of reported deaths are due to severe bleeding complications. Aubron et al. Critical Care, 2013, 17:R73 looked at the factors associated with ECMO outcomes. The compounds of the present invention, which can be used as, for example, Factor XIa inhibitors, are thus contemplated as an alternative replacement for heparin in ECMO therapy. The compounds of the present invention are contemplated as effective agents for blocking the intrinsic pathway at plasma levels that will afford effective anti-coagulation/anti-thrombosis without marked bleeding liabilities. In some embodiments, the subject is sensitive to or has developed sensitivity to heparin. In some embodiments, the subject is resistant to or has developed resistance to heparin.

### Ischemia

“Ischemia” or an “ischemic event” is a vascular disease generally involving vascular occlusion or a restriction in blood supply to tissues. Ischemia can cause a shortage of oxygen and glucose needed for cellular metabolism. Ischemia is generally caused by problematic blood vessels that result in damage or dysfunction of tissue. Ischemia can also refer to a local loss in blood or oxygen in a given part of the body resulting from congestion (e.g., vasoconstriction, thrombosis, or embolism). Causes include embolism, thrombosis of an atherosclerosis artery, trauma, venous problems, aneurysm, heart conditions (e.g., myocardial infarction, mitral valve disease, chronic arterial fibrillation, cardiomyopathies, and prosthesis), trauma or traumatic injury (e.g., to an extremity producing partial or total vessel occlusion), thoracic outlet syndrome, atherosclerosis, hypoglycemia, tachycardia, hypotension, outside compression of a blood vessel (e.g., by a tumor), sickle cell disease, localized extreme cold (e.g., by frostbite), tourniquet application, glutamate receptor stimulation, arteriovenous malformations, rupture of significant blood vessels supplying a tissue or organ, and anemia.

A transient ischemic event generally refers to a transient (e.g., short-lived) episode of neurologic dysfunction caused by loss of blood flow (e.g., in the focal brain, spinal cord, or retinal) without acute infarction (e.g., tissue death). In some embodiments, the transient ischemic event lasts for less than 72 hours, 48 hours, 24 hours, 12 hours, 10 hours, 8 hours, 4 hours, 2 hours, 1 hour, 45 minutes, 30 minutes, 20 minutes, 15 minutes, 10 minutes, 5 minutes, 4 minutes, 3 minutes, 2 minutes, or 1 minute.

### Angioedema

Angioedema is the rapid swelling of the dermis, subcutaneous tissue, mucosa, and submucosal tissues. Angioedema is typically classified as either hereditary or acquired.

“Acquired angioedema” can be immunologic, non-immunologic, or idiopathic; caused by e.g., allergy, as a side effect of medications, e.g., ACE inhibitor medications.

“Hereditary angioedema” or “HAE” refers to a genetic disorder that results in acute periods of edema (e.g., swelling) that may occur in nearly all parts of the body, including the face, limbs, neck, throat, larynx, extremities, gastrointestinal tract, and genitalia. Attacks of HAE can often be life-threatening, with severity depending on the area affected, e.g., abdominal attacks may result in intestinal obstruction, while swelling of the larynx and upper airway can lead to asphyxiation. Pathogenesis of hereditary angioedema may be related to unopposed activation of the contact pathway by the initial generation of kallikrein or clotting factors (e.g., Factor XII).

Signs and symptoms include swelling, e.g., of the skill of the face, mucosa of the mouth or throat, and tongue. Itchiness, pain, decreased sensation in the affected areas, urticaria (i.e., hives), or stridor of the airway may also be a sign of angioedema. However, there can be no associated itch, or urticaria, e.g., in hereditary angioedema. HAE subjects can experience abdominal pain (e.g., abdominal pain lasting one to five days, abdominal attacks increasing a subject's white blood cell count), vomiting, weakness, watery diarrhea, or rash. Bradykinin plays an important role in angioedema, particularly hereditary angioedema. Bradykinin is released by various cell types in response to numerous different stimuli and is a pain mediator. Interfering with bradykinin production or degradation can lead to angioedema. In hereditary angioedema, continuous production of enzyme kallikrein can facilitate bradykinin formation. Inhibition of kallikrein can interfere with bradykinin production; and treat or prevent angioedema.

### Viral Respiratory Infection

Described herein are methods of treating a subject who has a viral respiratory infection, comprising administering to the subject a compound, or a pharmaceutically acceptable salt thereof, or a pharmaceutical composition disclosed herein. The subject may be at risk of a thromboembolic or thromboinflammatory complication. Thus, in an aspect, provided herein is a method of treating a subject who has a viral respiratory infection, the method comprising administering to the subject a therapeutically effective amount of a compound of formula (I):

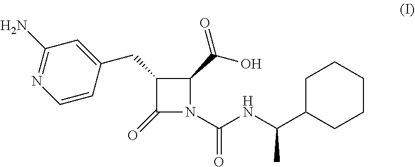 [SMILES: C[C@@H](NC(=O)N1C(=O)[C@H](Cc2ccnc(N)c2)[C@H]1C(=O)O)C1CCCCC1]

or a pharmaceutically acceptable salt thereof.

In some embodiments, the subject has (e.g., is identified as having) a risk of a thromboembolic or thromboinflammatory complication. In some embodiments, the subject is identified as having a viral respiratory infection. In some embodiments, the subject is identified as having a SARS-CoV-2 infection. In some embodiments, the subject is concurrently being treated for the SARS-CoV-2 infection. In some embodiments, the subject is in an intensive care setting or is identified as having symptom severity and/or general risk warranting admission to/care in an intensive care unit. In some embodiments, the subject is in a hospital setting (e.g., admitted to the hospital, in a hospital ward). In some embodiments, the subject has at least one D-dimer value greater than or equal to 1.0 μg/mL, e.g., within 24 hours of hospital admission. In some embodiments, the subject has at least one D-dimer value greater than or equal to 2 times local ULN, e.g., within 72 hours of hospital admission.

In another aspect, provided herein is a method of preventing thromboembolic or thromboinflammatory complications in a subject who has a viral respiratory infection, the method comprising administering to the subject a therapeutically effective amount of a compound of formula (I):

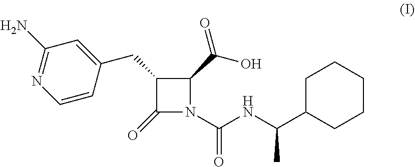 [SMILES: C[C@@H](NC(=O)N1C(=O)[C@H](Cc2ccnc(N)c2)[C@H]1C(=O)O)C1CCCCC1]

or a pharmaceutically acceptable salt thereof.

In some embodiments, the subject has (e.g., is identified as having) a risk of a thromboembolic or thromboinflammatory complication. In some embodiments, the subject is identified as having a viral respiratory infection. In some embodiments, the subject is identified as having a SARS-CoV-2 infection. In some embodiments, the subject is concurrently being treated for the SARS-CoV-2 infection. In some embodiments, the subject is in an intensive care setting or is identified as having symptom severity and/or general risk warranting admission to/care in an intensive care unit. In some embodiments, the subject is in a hospital setting (e.g., admitted to the hospital, in a hospital ward). In some embodiments, the subject has at least one D-dimer value greater than or equal to 1.0 μg/mL, e.g., within 24 hours of hospital admission. In some embodiments, the subject has at least one D-dimer value greater than or equal to 2 times local ULN, e.g., within 72 hours of hospital admission.

In another aspect, provided herein is a method of reducing the risk of thromboembolic or thromboinflammatory complications in a subject who has a viral respiratory infection, the method comprising administering to the subject a therapeutically effective amount of a compound of formula (I):

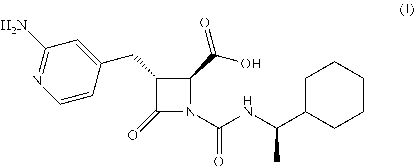 [SMILES: C[C@@H](NC(=O)N1C(=O)[C@H](Cc2ccnc(N)c2)[C@H]1C(=O)O)C1CCCCC1]

or a pharmaceutically acceptable salt thereof.

In some embodiments, the subject has (e.g., is identified as having) a risk of a thromboembolic or thromboinflammatory complication. In some embodiments, the subject is identified as having a viral respiratory infection. In some embodiments, the subject is identified as having a SARS-CoV-2 infection. In some embodiments, the subject is concurrently being treated for the SARS-CoV-2 infection. In some embodiments, the subject is in an intensive care setting or is identified as having symptom severity and/or general risk warranting admission to/care in an intensive care unit. In some embodiments, the subject is in a hospital setting (e.g., admitted to the hospital, in a hospital ward). In some embodiments, the subject has at least one D-dimer value greater than or equal to 1.0 ∥g/mL, e.g., within 24 hours of hospital admission. In some embodiments, the subject has at least one D-dimer value greater than or equal to 2 times local ULN, e.g., within 72 hours of hospital admission.

In some embodiments, the infection is caused by coronaviruses, rhinoviruses, respiratory syncytial viruses, or influenza viruses.

In some embodiments, the coronaviruses are SARS-CoV-1, SARS-CoV-2, MERS, or SARS, or mutated forms thereof.

In some embodiments, the coronaviruses is SARS-COV-2.

In some embodiments, the subject has a COVID-19 syndrome (e.g., identified as having a COVID-19 syndrome).

In some embodiments, the subject is identified as having an acute respiratory distress syndrome.

In some embodiments, the subject has undergone a renal replacement therapy.

In some embodiments, the subject has thrombosis of catheters or extracorporeal filters.

In some embodiments, the compound is administered prior to administration of a second agent (e.g., agent for treating or managing the viral respiratory infection).

In some embodiments, the compound is administered concomitantly with a second agent (e.g., agent for treating or managing the viral respiratory infection).

In some embodiments, wherein the compound is administered following administration of a second agent (e.g., agent for treating or managing the viral respiratory infection).

In some embodiments, the compound is administered intravenously.

In some embodiments, the compound is administered subcutaneously.

In some embodiments, the compound is administered as a continuous intravenous infusion.

In some embodiments, the compound is administered as a bolus.

In some embodiments, about 0.01 mg/kg/hr to about 10.0 mg/kg/hr of the compound is administered to the subject. In some embodiments, about 0.01 mg/kg/hr to about 5.0 mg/kg/hr of the compound is administered to the subject. In some embodiments, about 0.01 mg/kg/hr to about 4.0 mg/kg/hr of the compound is administered to the subject. In some embodiments, about 0.01 mg/kg/hr to about 3.0 mg/kg/hr of the compound is administered to the subject. In some embodiments, about 0.01 mg/kg/hr to about 2.0 mg/kg/hr of the compound is administered to the subject. In some embodiments, about 0.05 mg/kg/hr to about 5.0 mg/kg/hr of the compound is administered to the subject. In some embodiments, about 0.05 mg/kg/hr to about 4.0 mg/kg/hr of the compound is administered to the subject. In some embodiments, about 0.05 mg/kg/hr to about 3.0 mg/kg/hr of the compound is administered to the subject. In some embodiments, about 0.05 mg/kg/hr to about 2.0 mg/kg/hr of the compound is administered to the subject. In some embodiments, about 0.05 mg/kg/hr to about 1.0 mg/kg/hr of the compound is administered to the subject. In some embodiments, about 0.1 mg/kg/hr to about 5.0 mg/kg/hr of the compound is administered to the subject. In some embodiments, about 0.1 mg/kg/hr to about 4.0 mg/kg/hr of the compound is administered to the subject. In some embodiments, about 0.1 mg/kg/hr to about 3.0 mg/kg/hr of the compound is administered to the subject. In some embodiments, about 0.1 mg/kg/hr to about 2.0 mg/kg/hr of the compound is administered to the subject. In some embodiments, about 0.1 mg/kg/hr to about 1.0 mg/kg/hr of the compound is administered to the subject. In some embodiments, about 0.5 mg/kg/hr to about 5.0 mg/kg/hr of the compound is administered to the subject. In some embodiments, about 0.5 mg/kg/hr to about 4.0 mg/kg/hr of the compound is administered to the subject. In some embodiments, about 0.5 mg/kg/hr to about 3.0 mg/kg/hr of the compound is administered to the subject. In some embodiments, about 0.5 mg/kg/hr to about 2.0 mg/kg/hr of the compound is administered to the subject. In some embodiments, about 0.5 mg/kg/hr to about 1.0 mg/kg/hr of the compound is administered to the subject. In some embodiments, about 0.6 mg/kg/hr to about 1.0 mg/kg/hr of the compound is administered to the subject.

In some embodiments, about 0.05 mg/kg/hr of the compound is administered to the subject. In some embodiments, about 0.1 mg/kg/hr of the compound is administered to the subject. In some embodiments, about 0.2 mg/kg/hr of the compound is administered to the subject. In some embodiments, about 0.3 mg/kg/hr of the compound is administered to the subject. In some embodiments, about 0.4 mg/kg/hr of the compound is administered to the subject. In some embodiments, about 0.5 mg/kg/hr of the compound is administered to the subject. In some embodiments, about 0.6 mg/kg/hr of the compound is administered to the subject. In some embodiments, about 0.7 mg/kg/hr of the compound is administered to the subject. In some embodiments, about 0.8 mg/kg/hr of the compound is administered to the subject. In some embodiments, about 0.9 mg/kg/hr of the compound is administered to the subject. In some embodiments, about 1.0 mg/kg/hr of the compound is administered to the subject. In some embodiments, about 1.1 mg/kg/hr of the compound is administered to the subject. In some embodiments, about 1.2 mg/kg/hr of the compound is administered to the subject. In some embodiments, about 1.3 mg/kg/hr of the compound is administered to the subject. In some embodiments, about 1.4 mg/kg/hr of the compound is administered to the subject. In some embodiments, about 1.5 mg/kg/hr of the compound is administered to the subject. In some embodiments, about 1.6 mg/kg/hr of the compound is administered to the subject. In some embodiments, about 1.7 mg/kg/hr of the compound is administered to the subject. In some embodiments, about 1.8 mg/kg/hr of the compound is administered to the subject. In some embodiments, about 1.9 mg/kg/hr of the compound is administered to the subject. In some embodiments, about 2 mg/kg/hr of the compound is administered to the subject. In some embodiments, about 3 mg/kg/hr of the compound is administered to the subject. In some embodiments, about 4 mg/kg/hr of the compound is administered to the subject. In some embodiments, about 5 mg/kg/hr of the compound is administered to the subject.

In some embodiments, the complication is pulmonary embolism, deep vein thrombosis, a major bleeding episode, stroke, arterial thromboembolism, or ischemic stroke.

In some embodiments, the complication is pulmonary embolism, deep vein thrombosis, or a major bleeding episode.

In some embodiments, the complication is a blood clot.

In some embodiments, the complication is a cytokine response or coagulopathy.

In some embodiments, the subject has a reduced D-dimer level subsequent to administration of the compound (e.g., Compound 1 or a pharmaceutically acceptable salt thereof) to the subject relative to a subject not administered the compound (e.g., Compound 1 or a pharmaceutically acceptable salt thereof).

In some embodiments, the compound is administered to the subject before, during, or after a medical procedure.

In some embodiments, the medical procedure is comprises one or more of i) a cardiopulmonary bypass, ii) oxygenation and pumping of blood via extracorporeal membrane oxygenation, iii) assisted pumping of blood (internal or external), iv) dialysis of blood, v) extracorporeal filtration of blood, vi) collection of blood from the subject in a repository for later use in an animal or a human subject, vii) use of venous or arterial intraluminal catheter(s), viii) use of device(s) for diagnostic or interventional cardiac catherisation, ix) use of intravascular device(s), x) use of artificial heart valve(s), and xi) use of artificial graft(s).

In some embodiments, the medical procedure comprises an oxygenation and pumping of blood via extracorporeal membrane oxygenation (ECMO). For example, ECMO is venovenous ECMO or venoarterial ECMO.

In some embodiments, the subject is being or has been treated with a mechanical ventilation.

### Pharmaceutical Compositions

The compositions described herein include the compound described herein (e.g., Compound 1 as well as additional therapeutic agents, if present, in amounts effective for achieving the treatment of a disease or disease symptoms (e.g., such as a disease associated with Factor XIa).

Pharmaceutically acceptable carriers, adjuvants and vehicles that may be used in the pharmaceutical compositions provided herewith include, but are not limited to, ion exchangers, alumina, aluminum stearate, lecithin, self-emulsifying drug delivery systems (SEDDS) such as d-β-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions may be in the form of a solid lyophilized composition that can be reconstituted by addition of a compatible reconstitution diluent prior to parenteral administration or in the form of a frozen composition adapted to be thaws and, if desired, diluted with a compatible diluent prior to parenteral administration. In some embodiments, the pharmaceutical composition includes a powder (e.g. lyophilized composition) dissolved in aqueous medium, e.g., a saline solution, in a unit dosage IV bag or bottle at a concentration suitable for intravenous administration to a subject. In some embodiments, ingredients of a pharmaceutical composition suitable for intravenous administration are separated from each other in a single container, e.g., a powder comprising a compound described herein or a pharmaceutically acceptable salt thereof, is separated from an aqueous medium such as a saline solution. In this latter example, the various components are separated by a seal that can be broken to contact the ingredients with each other to form the pharmaceutical composition suitable for intravenous administration.

### Routes of Administration

The pharmaceutical compositions provided herewith may be administered orally, rectally, or parenterally (e.g., intravenous infusion, intravenous bolus injection, inhalation, implantation). The term parenteral as used herein includes subcutaneous, intracutaneous, intravenous (e.g., intravenous infusion, intravenous bolus injection), intranasal, inhalation, pulmonary, transdermal, intramuscular, intraarticular, intraarterial, intrasynovial, intrasternal, intrathecal, intralesional and intracranial injection or other infusion techniques. The pharmaceutical compositions provided herewith may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

The pharmaceutical compositions may be in the form of a sterile injectable preparation, for example, as a sterile injectable aqueous or oleaginous solution or suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans or other similar emulsifying agents or bioavailability enhancers which are commonly used in the manufacture of pharmaceutically acceptable solid, liquid, or other dosage forms may also be used for the purposes of formulation.

The pharmaceutical compositions provided herewith may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient may be suspended or dissolved in an oily phase is combined with emulsifying or suspending agents. If desired, certain sweetening or flavoring or coloring or taste masking agents may be added.

The compounds described herein can, for example, be administered by injection, intravenously (e.g., intravenous infusion, intravenous bolus injection), intraarterially, subdermally, intraperitoneally, intramuscularly, or subcutaneously; or orally, buccally, nasally, transmucosally, topically with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions provided herewith will be administered from about 1 to about 6 times per day (e.g., by intravenous bolus injection) or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

In some embodiments, a pharmaceutical composition formulated for oral administration, subcutaneous administration, or intravenous administration is administered to a subject from 1 time per day to 6 times per day (e.g., 2 times per day or 4 times per day). In some embodiments, a pharmaceutical composition formulated for oral administration is administered to a subject from 1 time per day to 6 times per day (e.g., 2 times per day or 4 times per day) for about 3 to 9 months. In some embodiments, a pharmaceutical composition formulated for oral administration is administered to a subject from 1 time per day to 6 times per day (e.g., 2 times per day or 4 times per day) for about 1 year. In some embodiments, a pharmaceutical composition formulated for oral administration is administered to a subject from 1 time per day to 6 times per day (e.g., 2 times per day or 4 times per day) for the rest of his or her life.

In some embodiments, the compound or pharmaceutical composition is administered to the subject intravenously. In some embodiments, the compound or pharmaceutical composition is administered to the subject subcutaneously. In some embodiments, the compound or pharmaceutical composition is administered to the subject as a continuous intravenous infusion. In some embodiments, the compound is administered to the subject as a bolus. In some embodiments, the compound or pharmaceutical composition is administered to the subject as a bolus followed by a continuous intravenous infusion.

### Combinations

In carrying out the methods of the present invention, it may be desired to administer the compounds of the invention (e.g., Factor XIa inhibitors) in combination with each other and one or more other agents for achieving a therapeutic benefit such as antithrombotic or anticoagulant agents, anti-hypertensive agents, anti-ischemic agents, anti-arrhythmic agents, platelet function inhibitors, and so forth. For example, the methods of the present invention may be carried out by administering the small molecule Factor XIa inhibitors in combination with a small molecule Factor XIa inhibitor. More particularly, the inventive methods may be carried out by administering the small molecule Factor XIa inhibitors in combination with aspirin, clopidogrel, ticlopidine or CS-747, warfarin, low molecular weight heparins (such as LOVENOX), GPIIb/GPIIIa blockers, PAI-1 inhibitors such as XR-330 and T-686, P2Y1 and P2Y12 receptor antagonists; thromboxane receptor antagonists (such as ifetroban), prostacyclin mimetics, thromboxane A synthetase inhibitors (such as picotamide), serotonin-2-receptor antagonists (such as ketanserin); compounds that inhibit other coagulation factors such as FVII, FVIII, FIX, FX, prothrombin, TAFI, and fibrinogen, or other compounds that inhibit FXI or kallikrein; fibrinolytics such as TPA, streptokinase, PAI-1 inhibitors, and inhibitors of □-2-antiplasmin such as anti-□-2-antiplasmin antibody fibrinogen receptor antagonists, inhibitors of □-1-antitrypsin, hypolipidemic agents, such as HMG-CoA reductase inhibitors (e.g., pravastatin, simvastatin, atorvastatin, fluvastatin, cerivastatin, AZ4522, and itavastatin), and microsomal triglyceride transport protein inhibitors (such as disclosed in U.S. Pat. Nos. 5,739,135, 5,712,279 and 5,760,246); antihypertensive agents such as angiotensin-converting enzyme inhibitors (e.g., captopril, lisinopril or fosinopril); angiotensin-II receptor antagonists (e.g., irbesartan, losartan or valsartan); ACE/NEP inhibitors (e.g., omapatrilat and gemopatrilat); or □-blockers such as propranolol, nadolol and carvedilol). The inventive methods may be carried out by administering the small molecule Factor XIa inhibitors in combination with anti-arrhythmic agents such as for atrial fibrillation, for example, amiodarone or dofetilide. The inventive methods may also be carried out in combination continuous renal replacement therapy for treating, e.g., acute kidney injury.

In carrying out the methods of the present invention, it may be desired to administer the compounds of the invention (Factor XIa inhibitors) in combination with agents that increase the levels of cAMP or cGMP in cells for a therapeutic benefit. For example, the compounds of the invention may have advantageous effects when used in combination with phosphodiesterase inhibitors, including PDE1 inhibitors (such as those described in Journal of Medicinal Chemistry, Vol. 40, pp. 2196-2210 [1997]), PDE2 inhibitors, PDE3 inhibitors (such as revizinone, pimobendan, or olprinone), PDE4 inhibitors (such as rolipram, cilomilast, or piclamilast), PDE7 inhibitors, or other PDE inhibitors such as dipyridamole, cilostazol, sildenafil, denbutyline, theophylline (1,2-dimethylxanthine), ARIFLOT.TM. (i.e., cis-4-cyano-4-[3 -(cyclopenlylox-y)-4-methoxyphenyl]cyclohexane- 1-carboxyl-ic acid), arofyline, roflumilast, C-11294A, CDC-801, BAY-19-8004, cipamfylline, SCH351591, YM-976, PD-189659, mesiopram, pumafentrine, CDC-998, IC-485, and KW-4490.

The inventive methods may be carried out by administering the compounds of the invention in combination with prothrombolytic agents, such as tissue plasminogen activator (natural or recombinant), streptokinase, reteplase, activase, lanoteplase, urokinase, prourokinase, anisolated streptokinase plasminogen activator complex (ASPAC), animal salivary gland plasminogen activators, and the like.

The inventive methods may be carried out by administering the compounds of the invention in combination with β-adrenergic agonists such as albuterol, terbutaline, formoterol, salmeterol, bitolterol, pilbuterol, or fenoterol; anticholinergics such as ipratropium bromide; anti-inflammatory cortiocosteroids such as beclomethasone, triamcinolone, budesonide, fluticasone, flunisolide or dexamethasone; and anti-inflammatory agents such as cromolyn, nedocromil, theophylline, zileuton, zafirlukast, monteleukast and pranleukast.

Small molecule Factor XIa inhibitors may act synergistically with one or more of the above agents. Thus, reduced doses of thrombolytic agent(s) may be used, therefore obtaining the benefits of administering these compounds while minimizing potential hemorrhagic and other side effects.

### Course of Treatment

The compositions described herein include an effective amount of a compound of the invention (e.g., a Factor XIa inhibitor) in combination and one or more other agents (e.g., an additional therapeutic agent) such as antithrombotic or anticoagulant agents, anti-hypertensive agents, anti-ischemic agents, anti-arrhythmic agents, platelet function inhibitors, and so forth for achieving a therapeutic benefit.

In some embodiments, the additional therapeutic agent is administered following administration of the compound of the invention (e.g., a Factor XIa inhibitor). In some embodiments, the additional therapeutic agent is administered 15 minutes, 30 minutes, 1 hour, 2 hours, 4 hours, 6 hours, 8 hours, 10 hours, 12 hours, 14 hours, 18 hours, 24 hours, 48 hours, 72 hours or longer after administration of the compound of the invention (e.g., a Factor XIa inhibitor). In some embodiments, the additional therapeutic agent is administered (e.g., orally) after discharge from a medical facility (e.g., a hospital).

In some embodiments, the compound of the invention (e.g., a Factor XIa inhibitor) and the additional therapeutic agent are co-formulated into a single composition or dosage. In some embodiments, the compound of the invention (e.g., a Factor XIa inhibitor) and the additional therapeutic agent are administered separately. In some embodiments, the compound of the invention (e.g., a Factor XIa inhibitor) and the additional therapeutic agent are administered sequentially. In some embodiments, the compound of the invention (e.g., a Factor XIa inhibitor) and the additional therapeutic agent are administered separately and sequentially. In general, at least one of the compound of the invention (e.g., a Factor XIa inhibitor) and the additional therapeutic agent is administered parenterally (e.g., intranasally, intramuscularly buccally, inhalation, implantation, transdermal, intravenously (e.g., intravenous infusion, intravenous bolus injection), subcutaneous, intracutaneous, intranasal, pulmonary, transdermal, intraarticular, intraarterial, intrasynovial, intrasternal, intrathecal, intralesional and intracranial injection or other infusion techniques); orally; or rectally, for example, intramuscular injection or intravenously (e.g., intravenous infusion, intravenous bolus injection)). In some embodiments, compound of the invention is administered parenterally (e.g., intranasally, buccally, intravenously (e.g., intravenous infusion, intravenous bolus injection) or intramuscularly). In some embodiments, the additional therapeutic agent is administered orally. In some embodiments, the compound of the invention (e.g., a Factor XIa inhibitor) is administered parenterally (e.g., intranasally, buccally, intravenously (e.g., intravenous infusion, intravenous bolus injection) or intramuscularly) and the additional therapeutic agent is administered orally.

In some embodiments, the compound of the invention (e.g., a Factor XIa inhibitor) may be administered once or several times a day. A duration of treatment may follow, for example, once per day for a period of about 1, 2, 3, 4, 5, 6, 7 days or more. In some embodiments, the treatment is chronic (e.g., for a lifetime). In some embodiments, either a single dose in the form of an individual dosage unit or several smaller dosage units or by multiple administrations of subdivided dosages at certain intervals is administered. For instance, a dosage unit can be administered from about 0 hours to about 1 hr, about 1 hr to about 24 hr, about 1 to about 72 hours, about 1 to about 120 hours, or about 24 hours to at least about 120 hours post injury. Alternatively, the dosage unit can be administered from about 0.5, 1, 1.5, 2, 3, 4, 5, 6, 7, 8, 9, 10, 11, 12, 13, 14, 15, 16, 17, 18, 19, 20, 21, 22, 23, 24, 30, 40, 48, 72, 96, 120 hours or longer post injury. Subsequent dosage units can be administered any time following the initial administration such that a therapeutic effect is achieved. In some embodiments, the initial dose is administered orally. In some embodiments, doses subsequent to the initial dose are administered parenterally (e.g., intranasally, intramuscularly buccally, inhalation, implantation, transdermal, intravenously (e.g., intravenous infusion, intravenous bolus injection), subcutaneous, intracutaneous, intranasal, pulmonary, transdermal, intraarticular, intraarterial, intrasynovial, intrasternal, intrathecal, intralesional and intracranial injection or other infusion techniques); orally; or rectally.

In some embodiments, compounds of the invention (e.g., a Factor XIa inhibitor) is administered orally, e.g., as an liquid or solid dosage form for ingestion, for about 5 minutes to about 1 week; about 30 minutes to about 24 hours, about 1 hour to about 12 hours, about 2 hours to about 12 hours, about 4 hours to about 12 hours, about 6 hours to about 12 hours, about 6 hours to about 10 hours; about 5 minutes to about 1 hour, about 5 minutes to about 30 minutes; about 12 hours to about 1 week, about 24 hours to about 1 week, about 2 days to about 5 days, or about 3 days to about 5 days. In one embodiment, the compound of the invention (e.g., a Factor XIa inhibitor) is administered orally as a liquid dosage form. In another embodiment, the compound of the invention (e.g., a Factor XIa inhibitor) is administered orally as a solid dosage form.

Where a subject undergoing therapy exhibits a partial response, or a relapse following completion of the first cycle of the therapy, subsequent courses of therapy may be needed to achieve a partial or complete therapeutic response (e.g., chronic treatment, e.g., for a lifetime).

In some embodiments, the compound of the invention (e.g., a Factor XIa inhibitor) is administered intravenously, e.g., as an intravenous infusion or intravenous bolus injection, for about 5 minutes to about 1 week; about 30 minutes to about 24 hours, about 1 hour to about 12 hours, about 2 hours to about 12 hours, about 4 hours to about 12 hours, about 6 hours to about 12 hours, about 6 hours to about 10 hours; about 5 minutes to about 1 hour, about 5 minutes to about 30 minutes; about 12 hours to about 1 week, about 24 hours to about 1 week, about 2 days to about 5 days, or about 3 days to about 5 days. In one embodiment, the compound of the invention (e.g., a Factor XIa inhibitor) is administered as an intravenous infusion for about 5, 10, 15, 30, 45, or 60 minutes or longer; about 1, 2, 4, 6, 8, 10, 12, 16, or 24 hours or longer; about 1, 2, 3, 4, 5, 6, 7, 8, 9, or 10 days or longer.

### Dosages and Dosing Regimens

The effective amount of a small molecule Factor XIa inhibitor administered according to the present invention may be determined by one of ordinary skill in the art. The specific dose level and frequency of dosage for any particular subject may vary and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

Upon improvement of a patient's condition, a maintenance dose of a compound, composition or combination provided herewith may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

### EXAMPLES

In order that the invention described herein may be more fully understood, the following examples are set forth. Starting materials and various intermediates described in the following examples may be obtained from commercial sources, prepared from commercially available organic compounds, or prepared using known synthetic methods. The examples described in this application are offered to illustrate the compounds provided herein and are not to be construed in any way as limiting their scope.

### Example 1: Compound 1 for Prevention of Thromboembolic Complications in Critically Ill SARS-CoV-2 Patients

In the study, Compound 1 will be investigated for prevention of thrombotic and bleeding complications in patients managed in the intensive care unit (ICU) for COVID-19 syndrome. The patients will be administered Compound 1 as a continuous intravenous infusion at 0.6 mg/kg/hr or 1.0 mg/kg/hr, e.g. for the duration of the index hospitalization. Patients in this study are identified as being positive (e.g., PCR-confirmed) for SARS-CoV-2 viral infection (COVID-19 syndrome). Moreover, the patients in the study are being treated in an intensive care setting (e.g., at the intensive care unit (ICU)). Furthermore, patients in the study may have one D-dimer value≥1.0 μg/mL within 24 hours of hospital admission or one D-dimer value≥2 times local ULN within 72 hours of hospital admission and clinical condition (e.g., by symptom severity and/or general risk) warranting ICU admission within 24 hours of start of study drug. Primary objective of this study is to identify the appropriate dose of Compound 1 that demonstrates safety and tolerability of Compound 1 in the prevention of thromboembolism in the management of COVID-19 patients throughout a hospital stay and the post-hospital (e.g., 30 days post-hospital, after discharge), for example, in comparison to patients managed with institutional standard care thromboprophylaxis regimens.

Primary endpoint is an ISTH-defined major bleeding episode occurring until index hospital discharge. Additional endpoints include patient all-cause mortality as a single endpoint, mortality directly attributable to Venous Thromboembolism (VTE) as a single endpoint, Pulmonary Embolism (PE) as a single endpoint, Deep Venous Thrombosis (DVT) as a single endpoint, stroke as a single endpoint, results of any diagnostic or prognostic laboratory studies aimed at assessing coagulation status, results of diagnostic imaging studies for the evaluation of possible VTE or stroke, incidence of clinically relevant nonmajor bleeding, number of patients receiving a packed red blood cell infusion, number of patients receiving FFP, PCC, cryoprecipitate, or tranexamic acid. Other endpoints may include: comparative efficacy as measured by time to a thrombotic or thromboembolic event with Compound 1 compared to institutional standard thromboprophylaxis, assessed at the time of index hospital discharge (where an “event” is defined as inclusive of (1) death, (2) diagnosed symptomatic pulmonary embolism, (3) diagnosed symptomatic deep vein thrombosis, (4) symptomatic diagnosed arterial thromboembolism, or (5) symptomatic ischemic stroke, occurring until index hospital discharge), days in ICU, hospital length of stay, trends in D-dimer level, nadir hemoglobin, nadir platelet count, serial levels of other primary inflammatory markers of COVID-19, including LDH, ferritin, C-reactive protein (CRP), and interleukin-6 (IL-6), measures of healthcare resource consumption (HEOR), time to an efficacy event (defined above) between index hospital discharge and 30 days, stratified by post-discharge antithrombotic therapy, and time to a safety event (defined above) between index hospital discharge and 30 days, stratified by post-discharge antithrombotic therapy.

Dosing of Compound 1 for the first 30 patients enrolled will be randomized 1:1 to 0.6 mg/kg/hr IV (n=15) or 1.0 mg/kg/hr IV (n=15) for the duration of the index hospitalization. Enrollment will be paused after treatment of these 30 patients, at which time a dedicated DSMB will evaluate all collected safety data through 7 days post-index hospital discharge. If no adverse safety signal is identified for the 1.0 mg/kg/hr dose, enrollment will be resumed with the study dose of Compound 1 randomized 2:1 to 1.0 mg/kg/hr or institutional standard care for thromboprophylaxis in ICU patients with COVID-19. If safety concerns are identified for the higher dose and not for the lower dose, then open-label randomized enrollment vs institutional standard of care for thromboprophylaxis will proceed with a Compound 1 dose of 0.6 mg/kg/hr.

Inhibition of Factor XIa may represent a unique target to safely prevent and treat thromboinflammatory complications due to COVID-19, including the cytokine response and coagulopathy, and to reduce the associate mortality.

### Incorporation by Reference

The entire disclosure of International Patent Application Publications WO2015/120062, WO2020/092594, and WO2020/159824, and each of the patent documents and scientific articles referred to herein is incorporated by reference for all purposes.

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of treating a subject who has a viral respiratory infection, the method comprising administering to the subject a therapeutically effective amount of a compound of formula (I): 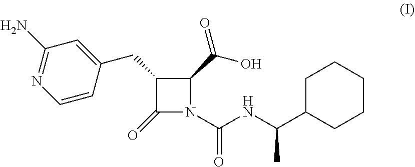 [SMILES: C[C@@H](NC(=O)N1C(=O)[C@H](Cc2ccnc(N)c2)[C@H]1C(=O)O)C1CCCCC1]
or a pharmaceutically acceptable salt thereof.

2. The method of claim 1, wherein the subject is at risk of a thromboembolic or thromboinflammatory complication.

3. A method of preventing thromboembolic or thromboinflammatory complications in a subject who has a viral respiratory infection, the method comprising administering to the subject a therapeutically effective amount of a compound of formula (I): 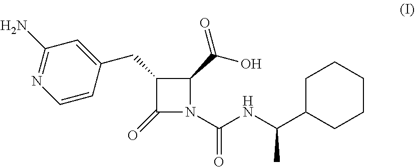 [SMILES: C[C@@H](NC(=O)N1C(=O)[C@H](Cc2ccnc(N)c2)[C@H]1C(=O)O)C1CCCCC1]
or a pharmaceutically acceptable salt thereof.

4. A method of reducing the risk of thromboembolic or thromboinflammatory complications in a subject who has a viral respiratory infection, the method comprising administering to the subject a therapeutically effective amount of a compound of formula (I): 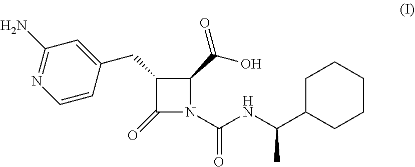 [SMILES: C[C@@H](NC(=O)N1C(=O)[C@H](Cc2ccnc(N)c2)[C@H]1C(=O)O)C1CCCCC1]
or a pharmaceutically acceptable salt thereof.

5. The method of any one of claims 1-4, wherein the infection is caused by coronaviruses, rhinoviruses, respiratory syncytial viruses, or influenza viruses.

6. The method of claim 5, wherein the coronaviruses are SARS-CoV-1, SARS-CoV-2, MERS, or SARS, or mutated forms thereof. 7 The method of claim 6, wherein the coronaviruses is SARS-COV-2.

8. The method of any one of claims 1-7, wherein the subject is identified as having a COVID-19 syndrome.

9. The method of any one of claims 1-8, wherein the compound is administered prior to administration of a second agent (e.g., agent for treating or managing the viral respiratory infection).

10. The method of any one of claims 1-8, wherein the compound is administered concomitantly with a second agent (e.g., agent for treating or managing the viral respiratory infection).

11. The method of any one of claims 1-8, wherein the compound is administered following administration of a second agent (e.g., agent for treating or managing the viral respiratory infection).

12. The method of any one of claims 1-11, wherein the compound is administered intravenously.

13. The method of any one of claims 1-11, wherein the compound is administered subcutaneously.

14. The method of any one of claims 1-11, wherein the compound is administered as a continuous intravenous infusion.

15. The method of any one of claims 1-11, wherein the compound is administered as a bolus.

16. The method of any one of claims 1-15, wherein about 0.6 mg/kg/hr to about 1.0 mg/kg/hr of the compound is administered to the subject.

17. The method of any one of claims 1-16, wherein the complication is pulmonary embolism, deep vein thrombosis, or a major bleeding episode.

18. The method of any one of claims 1-16, wherein the complication is a blood clot.

19. The method of any one of claims 1-18, wherein the compound is administered to the subject before, during, or after a medical procedure.

20. The method of claim 19, wherein the medical procedure is comprises one or more of i) a cardiopulmonary bypass, ii) oxygenation and pumping of blood via extracorporeal membrane oxygenation, iii) assisted pumping of blood (internal or external), iv) dialysis of blood, v) extracorporeal filtration of blood, vi) collection of blood from the subject in a repository for later use in an animal or a human subject, vii) use of venous or arterial intraluminal catheter(s), viii) use of device(s) for diagnostic or interventional cardiac catherisation, ix) use of intravascular device(s), x) use of artificial heart valve(s), and xi) use of artificial graft(s).

21. The method of claim 20, wherein the medical procedure comprises an oxygenation and pumping of blood via extracorporeal membrane oxygenation (ECMO).

22. The method of claim 21, wherein ECMO is venovenous ECMO or venoarterial ECMO.

23. The method of any one of claims 1-22, wherein the subject is being or has been treated with a mechanical ventilation.

24. The method of any one of claims 1-23, wherein the subject is at risk of thrombopenia.

25. The method of any one of claims 1-23, wherein the subject is identified as having thrombopenia.
